(19) United States
(12) Patent Application Publication
(10) Pub. No.: US 2010/0228260 A1
(43) Pub. Date: Sep. 9, 2010

(54) INJECTOR FOR INTRAOCULAR LENS

(75) Inventors: Wayne B. CALLAHAN, Abingdon, VA (US); Anna S. HAYES, Newton Centre, MA (US)

Correspondence Address: REMENICK PLLC, 1025 THOMAS JEFFERSON STREET, NW, WASHINGTON, DC, 20007, US

(73) Assignee: Anew Optics, Inc.

(21) Appl. No.: 12/717,748

(22) Filed: Mar. 4, 2010

Related U.S. Application Data
(60) Provisional application No. 61/157,361, filed on Mar. 4, 2009.

(51) Int. Cl. A61F 9/007; A61L 2/08

(52) U.S. Cl. 606/107; 422/26; 422/28

(57) ABSTRACT

The invention is directed to an injector assembly and method of using the injector assembly that is part of a package in which the lens is sterilized and shipped together with the injector. The assembly comprises an injector barrel which has a circular end and a tapered end, an intraocular lens, and a haptic within the barrel. The assembly is comprised of a material that is packaged and sterilized as a single unit.

### REFERENCE TO RELATED APPLICATIONS

This Application claims priority to U.S. Provisional Application No. 61/157,361 entitled “Injector for Intraocular Lens” filed Mar. 4, 2009, the entirety of which is hereby incorporated by reference.

### BACKGROUND

1. Field of the Invention

This invention is directed to injectors for intraocular lenses that provide for the injection of intraocular lens into the eye of a patient. In particular, the invention is directed to injectors and methods of safe and effective injection of lenses.

2. Description of the Background

Injectors for the insertion of intraocular lenses (IOL) into an eye are generally described as tapered with cantilevered fingers that are similar to a funnel (U.S. Pat. No. 5,123,905) or a cone, with a slot rolled into a channel for the lens (U.S. Pat. Nos. 5,425,734; 5,468,246; 5,643,275 and 5,772,667). Injectors are also described as a sleeve that transports the lens through the injector wherein the sleeve functions much like forceps to guide the lens through the injector barrel or lumen (U.S. Pat. No. 6,605,093). There are also several cartridge injectors that utilize cantilevered fingers to guide the compressed lens through the injector (U.S. Pat. Nos. 5,947,976 and 6,537,283).

One injector device uses a hinged substrate similar to a cartridge which folds or compresses the lens before placing it into an injector (U.S. Pat. No. 5,976,150). Therein are also a second cartridge injectors (U.S. Pat. Nos. 6.398.786 and 6,010,510), that hold an IOL and the injector (U.S. Pat. Nos. 6,129,723 and 6,447,519).

U.S. Pat. No. 6,203,549 discloses a lens in separate container that attaches to the injector nose with a plunger that is not a complete circle. U.S. Pat. No. 6,976,989 describes an injector that squeezes, rolls, or compresses the lens prior to movement of the plunger to inject the lens into the eye. U.S. Pat. Nos. 7,037,328 and 7,279,006 disclose an injector and lens combination where the lens is a single piece with tripod footplate-like haptics. The first haptic leads through the injector and the second two have flexible push rods against them to guide and force the lens through the injector. U.S. Pat. No. 6,203,549 discloses a plunger end-tip design. U.S. Pat. No. 6,605,093 discloses the process of squeezing and guiding the lens through a lumen of an injector. A need exists for an injector design that is safer and more efficient for use by the ophthalmologist than conventional designs, and minimizes any risk of pain, damage, infection and/or injury to the patient.

### SUMMARY OF THE INVENTION

The present invention overcomes the problems and, disadvantages associated with current strategies and designs, and provides new injector designs as well as methods for their manufacture and use.

One embodiment of the invention is directed to an injector assembly that is part of a package in which the lens is sterilized and shipped together with the injector. The assembly comprises an injector barrel which has a circular end and a tapered end; and an intraocular lens and a haptic within the barrel; wherein the assembly is comprised of a material that is packaged and sterilized as a single unit.

Another embodiment of the invention is directed to methods for inserting an intraocular lens into an eye of a patient,comprising: providing a sterilized assembly composed of an injector barrel which has a circular end and a tapered end, wherein the tapered end extends into the eye;, an intraocular lens within the barrel; a rod within the barrel wherein the rod has a thumb pad at one end and two protrusions at the other end; pushing on the thumb pad to move the lens through the barrel toward the tapered end and into the eye.

The another embodiment of the invention is directed to an injector that functions as a container for the lens in a sealed package designed for sterilization, such as, steam or gas. One end of the container is taped much like a cannula and is designed to accommodate a lens that has a 475 micron center thickness and 6 millimeter overall lens optic diameter and can be implanted through a 2.2 millimeter incision without compressing any of the water from the lens when using a material containing 18% water. A small amount of fluid is extracted when using higher water content materials. After placing the lens within the container, soft pliable stoppers are placed in each end; the container is sterilized, and then placed into a larger container with tolerances to prevent movement between the packages or the stoppers. At the time of use, the container holding the lens is removed from the outer package, fitted into an oriented injector sleeve, and the stoppers are removed from both ends. Next a viscoelastic material of high molecular weight is injected into both ends of the container that is now functioning as a syringe, in the narrow end as a lubricating material consistent with established surgical practices for lens implants, and in the wide end of the syringe barrel to assist in impelling the lens through the syringe. A rod is inserted into the syringe wherein a lens contact end is configured in a grooved gap to fit the lens surface, so as to prevent point loading of forces on the lens and concomitant large mass at the point where maximum cross sectional area of the lens is passing through the injector. Some distance from the end of the rod a plunger is attached to the rod. The tolerance between the plunger and inside of the syringe is such as to force the high molecular weight viscoelastic material to aid the plunger rod lens contact end-move the lens through the syringe, while mitigating undue pressure of the lens contact end on the edge of the lens. The lens is injected into the eye and the tip of the injector rod is used for positioning the lens within the eye.

Other embodiments and advantages of the invention are set forth in part in the description, which follows, and in part, may be obvious from this description, or may be learned from the practice of the invention.

### DESCRIPTION OF THE DRAWINGS

FIG. 1. Diagram of an embodiment of an injector-container.

FIG. 2. Diagram of an embodiment of an injector barrel cylinder end.

FIG. 3. Diagram of an embodiment of an intraocular lens—overhead view.

FIG. 4. Diagram of an embodiment of an intraocular lens—sagittal view.

FIG. 5. Diagram of an embodiment of an injector barrel—tapered end.

FIG. 6. Diagram of an embodiment of a stopper for injector end of barrel.

FIG. 7. Diagram of an embodiment of a stopper for tapered end of barrel.

FIG. 8. Diagram of an embodiment of an over sleeve for injector barrel.

FIG. 9. Diagram of an embodiment of an over sleeve tip.

FIG. 10. Diagram of an embodiment of a plunger rod for injector.

FIG. 11. Diagram of an embodiment of a plunger rod and injector assembly.

FIG. 12. Diagram of an embodiment of a tip of plunger rod.

FIG. 13. Diagram of an embodiment of a tip of plunger rod with lens engaged.

FIG. 14. Diagram of an embodiment of a lens in cylinder end of injector with clearance.

FIG. 15. Diagram of an embodiment of a lens in cylinder end without clearance.

FIG. 16. Diagram of an embodiment of a lens in barrel with forward movement.

FIG. 17. Diagram of an embodiment of a lens in barrel with additional forward movement.

FIG. 18. Diagram of an embodiment of a lens in barrel with initial movement of central optic.

FIG. 19. Diagram of an embodiment of a lens in barrel with additional movement of central optic.

FIG. 20. Diagram of an embodiment of a lens in barrel with additional movement of central optic.

FIG. 21. Diagram of an embodiment of a lens in barrel with most of the lens surfaces touching another lens surface.

FIG. 22. Diagram of an embodiment of a human eye cross section.

FIG. 23. Diagram of an embodiment of a human eye with natural lens and section of anterior capsule removed.

FIG. 24. Diagram of an embodiment of an injector with lens advanced to tapered tip end.

FIG. 25. Diagram of an embodiment of an injector with lens advanced magnified.

FIG. 26. Diagram of an embodiment of a human eye with first stage of lens injection.

FIG. 27. Diagram of an embodiment of a human eye with distal lens haptic compressed.

FIG. 28. Diagram of an embodiment of a human eye with lens being positioned using injector.

FIG. 29. Diagram of an embodiment of a human eye with implanted intraocular lens.

FIG. 30. Diagram of an embodiment of a lens with closed looped haptics.

FIG. 31. Diagram of lens with open looped haptics.

FIG. 32. Diagram of an embodiment of a lens with plate haptics

### DESCRIPTION OF THE INVENTION

As embodied and broadly described herein, the disclosures herein provide detailed embodiments of the invention. However, the disclosed embodiments are merely exemplary of the invention that may be embodied in various and alternative forms. Therefore, there is no intent that specific structural and functional details should be limiting, but rather the intention is that they provide a basis for the claims and as a representative basis for teaching one skilled in the art to variously employ the present invention.

Conventional injectors for intraocular lenses are designed as a simple sleeve for transporting an intraocular lens from a container into the eye of a patient. The physician is required to remove the lens from one package and the injector from another, and transfer the lens into the injector manually for injection. This creates a substantial risk of contamination and improper insertion of the lens into the injector and thus the eye. Either result would be extremely detrimental and possibly permanently damaging to the patient.

It has been surprisingly discovered that an injector can be designed as a cartridge that is part of a package in which the lens is sterilized and shipped. The cartridge both holds the lens for transportation and serves as the injector assembly. The injector is designed as a sealed container and is preferably composed of glass, plastic, or other suitable material that can withstand sterilization of the intraocular lens (e.g. steam, heat or gas sterilization). The container itself functions as an injector barrel for insertion of the lens into an eye. After sterilization the sealed product is packaged and shipped to a surgeon especially trained for the implantation of intraocular lenses. The packaging is removed leaving a broad end and a tapered end of the injector. The injector is then inserted, tapered end first, into a specifically oriented sleeve that insures that the lens will be positioned correctly within the eye. The outer sleeve of this injector preferably has a split at the tip (last ¾ millimeter) for ease of pushing the sleeve into the incision in the eye and allowing the lens to pass through a 2.2 millimeter incision in the cornea, expand to allow the lens to pass through into the eye, and position the lens definitively in its intended location. The sleeve portion is flatter than the incision and is opened by the compressed lens as it passes through the injector tip and allows for a smooth and straightforward insertion not achieved by the conventional injector. Preferably, a viscoelastic material is added to both ends of the injector barrel to function as an injector. A rod is placed into the larger open end of the injector barrel which functions to squeeze the lens into a tapered section permitting a reduction in the dimensions of the lens as it enters the eye and the size of the incision used to implant the lens within the eye. The rod is equipped with a grooved gap at the forward end to serve as a contact end to affix to the rear of the lens just short of the lens optical apex, having passed over the attached haptic and thinner portion of the lens at that end. The viscoelastic at the broad end of the injector is held in the injector by a plunger attached to the rod at some distance from the contact end that serves to impel the lens through the barrel minimizing unwanted distortive pressure on the lens. The preferred lens is designed with a 475 microns center apex and a 100 microns peripheral section, with an overall outer diameter of 6 mm. The injector system can be used with a standard lens with a closed loop haptic or, with slight modifications to the injector, with an open looped haptic or a plate lens.

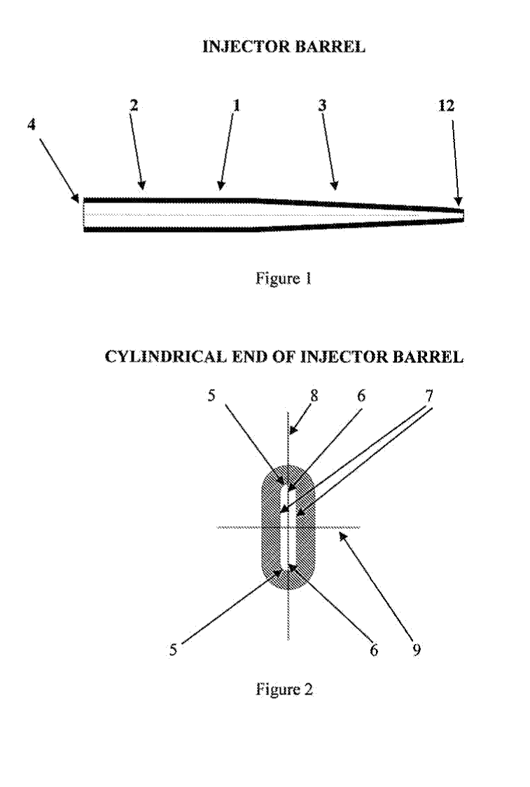

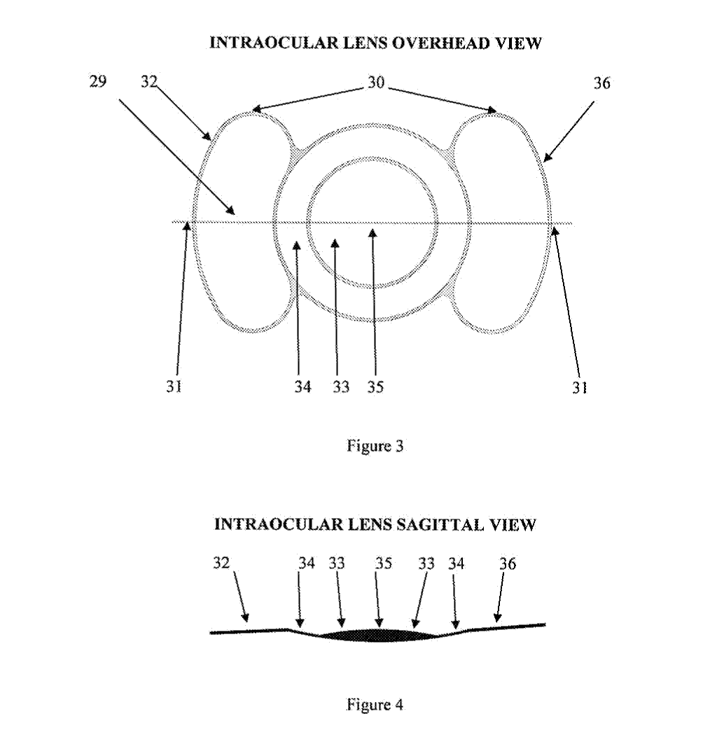

A preferred embodiment of the injector is described by reference to the figures. Part numbers of the depictions as set forth in the Figures are provided in the attached Table. The injector (FIG. 1) is preferably comprised of a barrel (1) with a cylindrical section (2) that is preferably 50 mm long, and a tapered section (3) that is 64 mm long. The cylindrical section (2) has an ovoidal cross section (4) (FIG. 2) with the minor axis sides (5) being an arc of a circle with a radius of 0.66 mm. The centers (6) of the minor axis side wall arcs (5) remain parallel throughout the length of the injector barrel (1). The sides (7) of the major axis (8) are parallel and equal in length. The major axis cross section is preferably 7 mm and bordered on each end by the minor axis side walls (5) with the apex of the side walls creating the largest opening which is of sufficient size to allow the widest point (30) of a lens (including haptics—part 32 and 36) (FIG. 3) to have a small amount of clearance between the side walls of the injector container and the lens. The widest width of the lens haptic is preferably 6.9 mm. The minor axis (9) is sufficiently large to allow the lens to move freely, but not to rotate enough to turn over, preferably 1.32 mm. The injector barrel holds the lens to where the longest length of the lens (31), preferably 11.2 mm, is parallel to the ovoidal cylindrical section (2) with parallel sides (7), and is of sufficient length to allow a push rod to be secured stably within the injector barrel (1), preferably 50 mm, and the grooved gap in the contact end of the rod to touch the proximate portion of the lens just short of the apex of the lens and push the lens far enough along the barrel (or lumen) to start folding the lens in the tapered section.

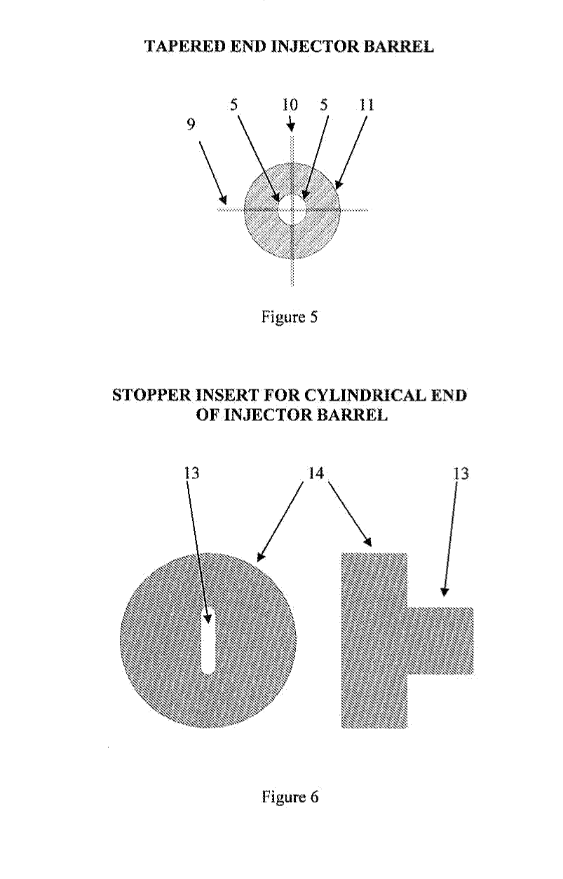

The second section of the injector barrel (1) is preferably 64 mm long and preferably a tapered section (3) allowing the lens to be compressed for insertion into the eye through a smaller incision. The sides (7) of the major axis of the tapered section (3) of the injector barrel remain parallel; however, the end arcs retain the same radii of 0.66 mm (FIG. 5 part 5). The major axis becomes smaller (10) as the distance from the parallel section of the injector-container increases. The tapered second section (3) preferably proves a taper angle of 2.5 degrees, 2.0 degrees, 1.5 degrees, 0.5 degrees or less. At the end of the, tapered section distal from the initial cylindrical opening the' major axis is reduced until the non-arced sides are preferably 0.14 mm, which is almost as small as the minor axis approximating the 0.66 radii circular end tip. Along both the cylindrical and the tapered sections the outside diameter of the injector barrel (1) is parallel to the inside diameter of the same section and 2 mm thick with-the exception of the last 6 millimeters of the tapered end (12) which is tapered, preferably to 6 degrees, to allow for an over sleeve to fit snugly and preferably with a luer taper.

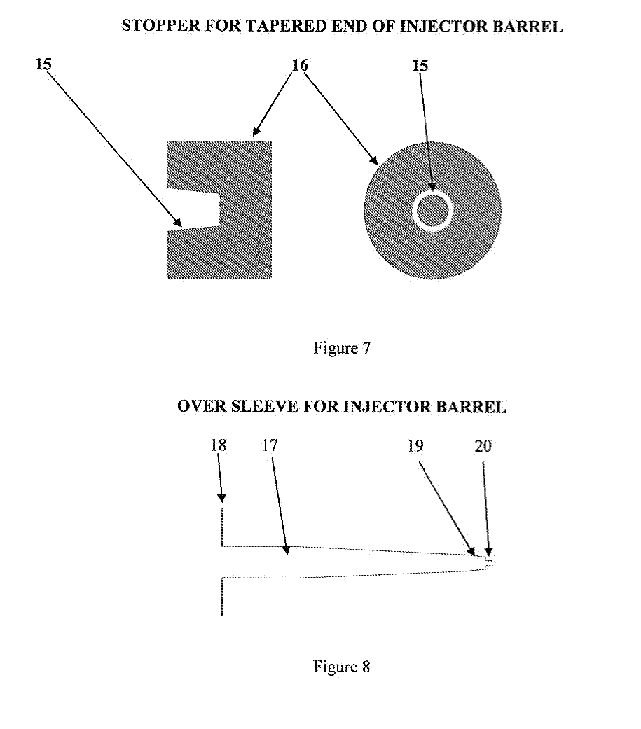

Prior to sterilization, a stopper (FIG. 6) with a protruding portion (13) contoured for a snug fit is inserted into the cylindrical (broad) end (4) of the injector barrel. The remainder of the stopper has a preferred diameter (14) of sufficient size to be slightly smaller than the internal diameter (14) of slightly less that 20 mm which is of sufficient size to be slightly smaller that the internal diameter of a second container that will house the injector barrel and assembled stoppers until ready for use by the surgeon or the medical practitioner. The second container can be any conventional vial used to package medical devices and/or pharmaceuticals. A second stopper (FIG. 7) tapered (15) to fit snuggly over the outside of the taper end of the barrel is positioned. The outside diameter (16) of the tapered end stopper is preferably slightly smaller than 20 mm, such as to allow the stopper to provide stability to the relationship between the injector barrel and the outer vial. The vial preferably has cylindrical inside walls and outside thread to secure an over stopper and cap conventionally used with such devices. Preferably, the outside diameter of the large stopper (14) and the outside diameter of the tapered stopper (16) are equal. The lens injector barrel can then be placed in a standard outer vial designed to be used with medical devices and pharmaceuticals for different sterilization methods. The second container can be any of several commercial vials used to package medical devices and pharmaceuticals. A standard sterile over wrap such as, for example, a TYVEK® pouch (pouch of flashspun synthetic high-density polyethylene fibers that is highly breathable although not permeable to liquid water) that is designed for the sterilization process can be placed around the sealed vial. After sterilization and final packaging the product is shipped to an ophthalmologist especially trained in the use of intraocular lenses.

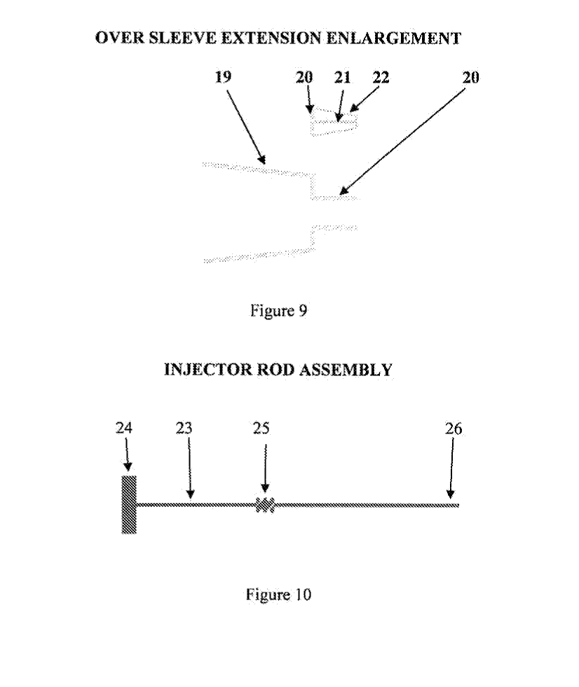

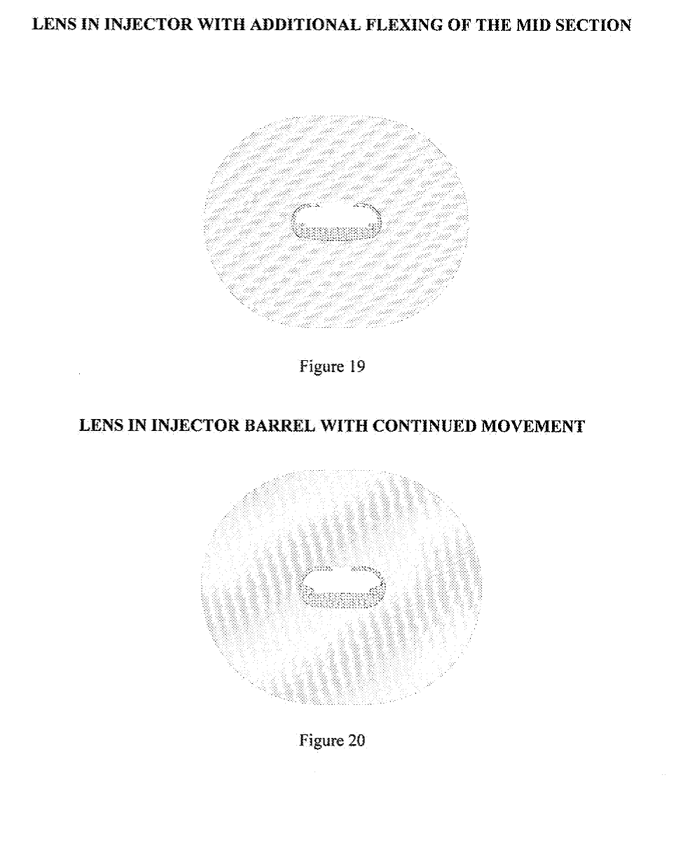

At the surgical location a nurse or other member of the surgical staff will remove the outer wrap, open the glass vial and remove the injector (1). The tapered stopper (15) is removed and the fluid in the container drained. An over sleeve (FIG. 8, part 17) made of a light weight plastic material with high mechanical ‘strength is placed over the injector barrel (1). The over sleeve is specifically oriented to accept the lens injector barrel such that the lens will be correctly positioned for insertion into the eye and the over sleeve slot end will slide correctly into the incision. The sleeve internal dimensions are slightly larger than the outside of the injector. The inner (5 and 7) and outer (11) dimensions of the injector are parallel except the last 6 millimeters of the injector barrel are tapered to 6 degrees to snuggly fit the taper in the end of the over sleeve. The inside of the tapered section of the injector barrel (10) is sloped to less than 3 degrees as the smaller the angle the less force needed to project the lens (29) through the barrel (lumen). With the exception of the last 6 millimeters, the outside (11) is parallel to the inside of the barrel. After the tapered section the over wrap is extended by 2.25 millimeters in length (FIG. 9-20) with a slot (21) cut along side and at the widest point of what has been the major axis. The slot is cut with a knife; therefore, very little material is removed and the slot has no width. The slot is designed to allow the over sleeve extension to easily penetrate the incision in the eye for the implantation of the lens. The over sleeve extension is 1.5 millimeters wide and the taper is 25 degrees starting with a dimension of 1.4 millimeters at the proximal end and tapered to 0.75 millimeters on the distal end which is the end making initial penetration into the incision in the eye. The only portion of the injector to actually contact the eye tissue is the slotted extension (20) of the over sleeve. As the lens progresses through the lumen of the barrel (1) and reaches the over wrap extension (20), the slot (21) opens to the maximum dimensions allowed by the incision in the eye. The lens passes through the incision and enters the eye.

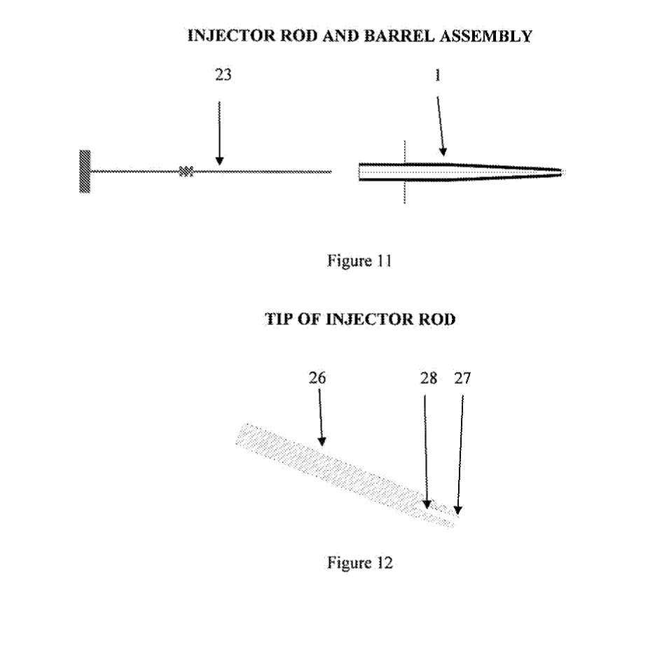

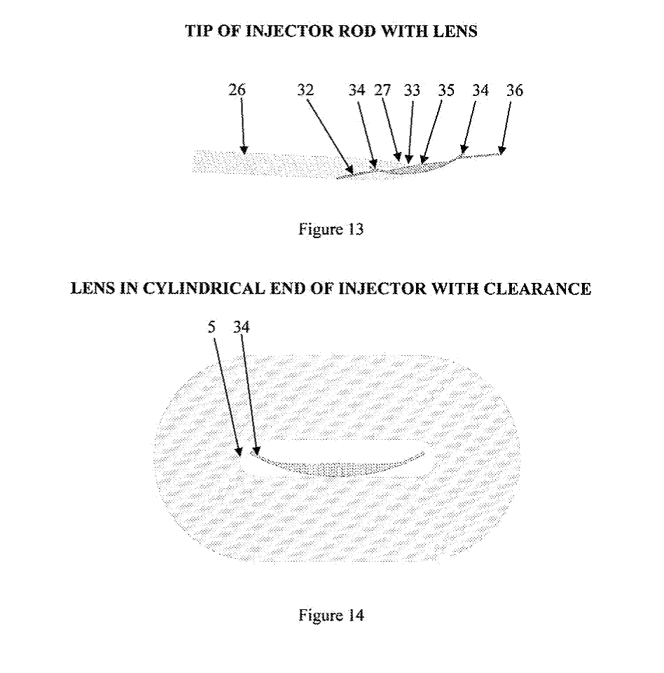

Preferably, the injector rod (FIG. 10, part 23) is comprised of an end thumb pad (24), with a compression gasket (25) preferably 50 mm along the rod from the thumb pad. The injector rod tip (23) with a preferred length of 135 mm is inserted into the cylindrical end (2) of the injector barrel (FIG. 11). The end distal to the thumb pad has an injector tip contact end (FIG. 12—part 26). The injector contact end (26) has a gap (28) of 400 microns and a length of 2.6 mm for the preferred lens. The leading edge (27) contacts the lens (FIG. 13) just short of the lens apex (35) and functions as forceps with a small space or gap of about 400 microns between the tips (28). Preferably, the tip of the injector rod does not connect with the distal haptic (36), but comes to rest against the optic (33) of the lens just short of the thinner outer section of the lens. Preferably, the lens is designed to where the center section (FIG. 13, part 35) is approximately 475 microns at the apex and the outer section (34) is 100 microns. The gap (28) is cut to about 2.6 mm to rest against the sides of the optic near the apex (35), but not close enough to the apex to occupy space along the critical cross section of the apex. The gap (28) is cut to allow the injector to slide over the thinner 100 micron end (34) of the lens optic and come to rest against the thicker optic (33) in a position where the rod tip leading edge (lens contact end) (27) will not obstruct the vertical space for the apex of the optic. The dimension of the rod across the minor axis of the barrel is 1.32 millimeters, which is the same as the minor axis of the barrel. The dimension of the rod in the plane of the major axis of the barrel is also 1.32 millimeters; which is 0.14 millimeters smaller than the major axis at the end of the tapered section.

The injector of the invention is preferably designed for use with lenses that are to be implanted in the natural lens envelope once the crystalline lens has been removed. The conditions that precipitate an ophthalmic surgeon's decision to remove the natural lens and replace it with an artificial lens made of poly-methyl methacrylate, hydrophilic acrylic material, hydrophobic acrylic material, silicone, or any other synthetic or natural material suitable for such lenses, encompass many diseases and conditions of the eye and include, inter-alia, myopia, hyperopia, presbyopia, and cataracts. In the first three instances, among others, the ophthalmic surgeon performs a clear lens replacement, removing the natural crystalline lens and installing an artificial lens, uniquely constructed to address the particular eye condition of the patient, in its place. When a patient suffers from cataracts, the natural lens (44) is cloudy not allowing adequate light to pass through, which reduces the patient's vision. Therefore, the natural lens is removed and replaced with an artificial intraocular lens. In other cases, the injector of the invention may be used to insert an artificial lens that is implanted to replace a prior artificial lens implant. In each of these cases the actual surgical procedure may vary, based upon the needs of the patient, the type of ophthalmic condition to be addressed, and the preference of the surgeon. The preferred methodology for the insertion of the lens by means of the injector is described below with the possible variation of the actual size of the surgical incision.

At the surgical location, a nurse or other member of the surgical staff removes the outer wrap while passing the sealed glass vial into the sterile field, then opens the glass vial and removes the injector barrel (1). Next a separate sterile package containing the outer sleeve and the plunger rod is opened and the sterile package passed into the sterile field. A second member of the surgical staff who is properly attired and has followed the necessary sterility procedures removes the assembled injector rod (23) and over sleeve (17) from the sterile wrap, then opens the lens vial and removes the injector barrel which contains the lens. The tapered stopper (15) is removed from the tapered end of the injector barrel, sterile fluid is emptied or drained from the narrow end and preferably replaced with viscoelastic, whereupon the over sleeve (17) is positioned around the injector barrel (1). The outer sleeve (FIG. 8, part 17) is preferably constructed of a plastic or other light weight material with high mechanical strength. Preferably, the sleeve internal dimensions are slightly larger than the outside of the injector barrel. The sleeve is oriented to provide finger grip tabs at the rear outer edges for ease of injection, with specific emphasis on correct positioning of the lens container within the sleeve for accurate placement in the eye.

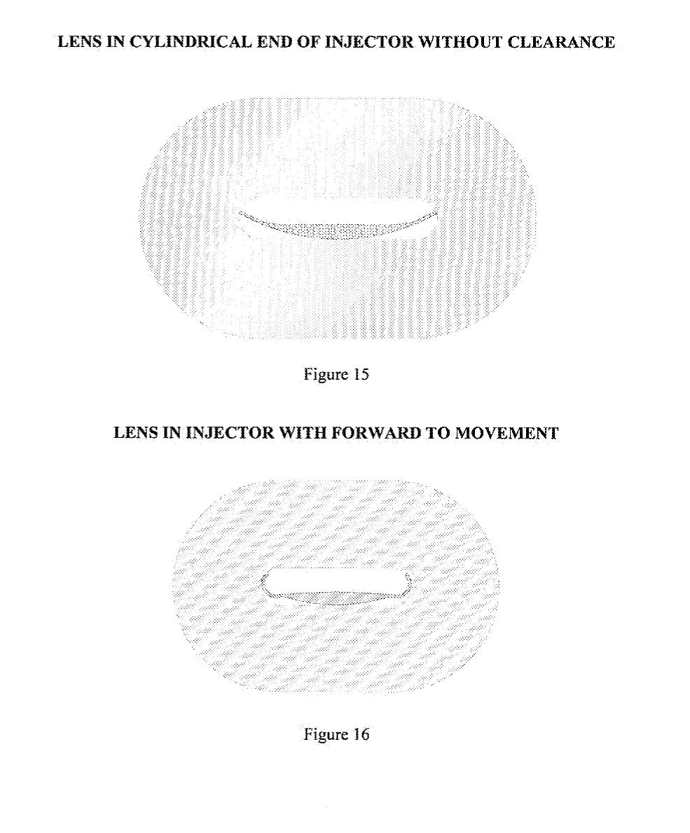

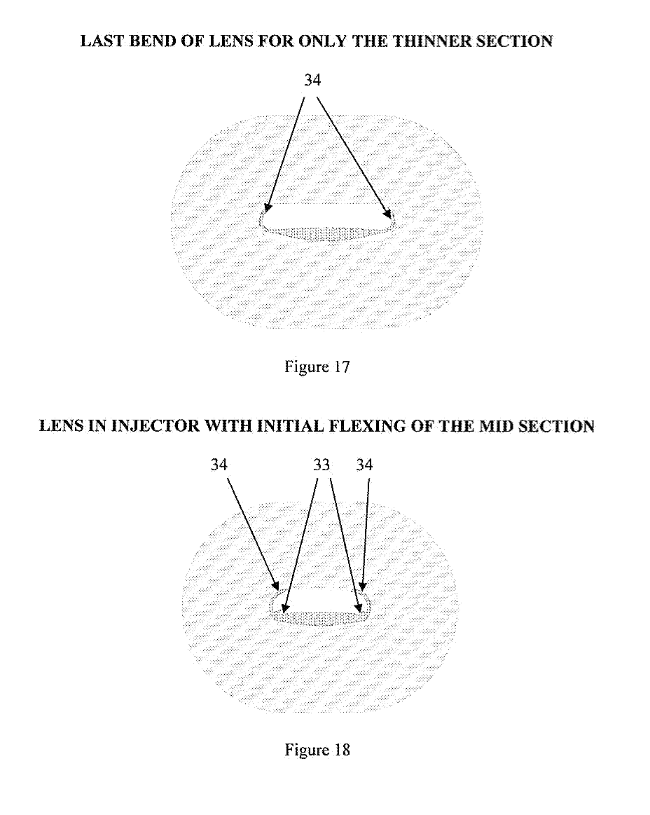

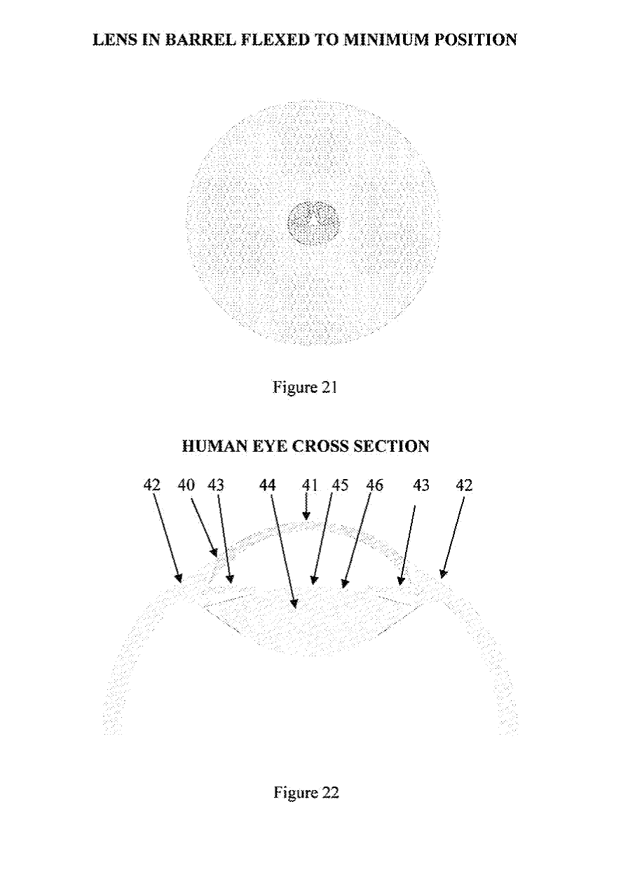

The surgical staff member removes the stopper (13) from the large cylindrical end, and injects viscoelastic into that end, thereafter the injector rod (23) placed into the cylindrical end (2) of the injector barrel. Care is taken to assure the tip leading edge (27) of the plunger rod tip (FIG. 13—part 27) does not contact the lens proximal haptic (32), but the haptic is placed under the contact end. The lens (29) is preferably shipped in the injector barrel (1) with clearance between the lens and the minor axis side walls (5) preferably as shown in FIG. 14. The surgical team member positions the lens in the injector by applying force to the thumb pad (24) on the end of the plunger rod (23) causing the lens (29) to move forward within the injector barrel. Initially, the movement of the lens within the plunger eliminates the clearance (FIG. 15) then only the outer thinner portion (100 microns) of the lens (34) is flexed (FIGS. 16 and 17), but as the barrel (lumen) becomes smaller the thicker middle section (33) of the lens between the thinner part and the apex (35) is squeezed. The process continues as shown in FIGS. 18 through 20 with FIG. 21 showing the lens fully compressed. The lens (29) is advanced though the injector barrel until the distal end tip of the distal haptic (36) can be seen, but none of the lens is physically in the over sleeve tip (20). When the lens (29) is fully flexed it has traveled to where the distal tip of the lens haptic (36) is at the end of the tapered section of the injector barrel (1). The lens is flexed, not compressed to where fluid is removed from the molecules, but confined to eliminate the space between the parts.

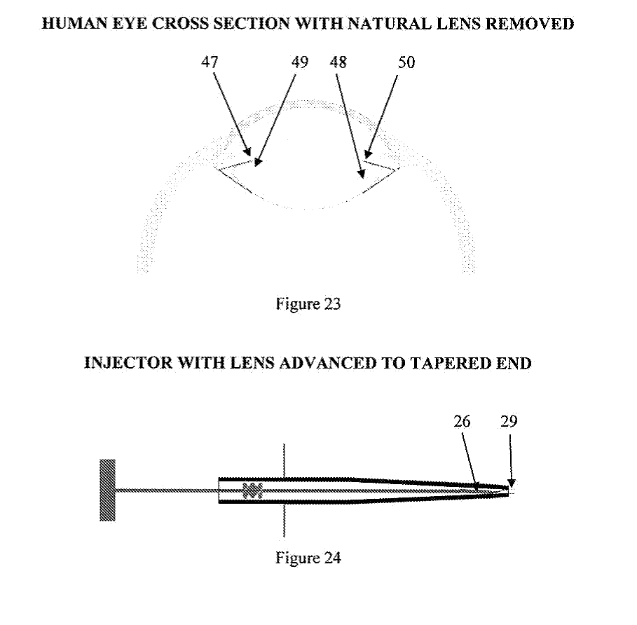

While the surgical team member is preparing the lens, the surgeon makes an incision (40) in the junction of the eye (FIG. 22) between the cornea (clear portion—part 41) and the sclera (white milky portion surrounding the cornea—part 42), preferably 2.2 mm and 200 microns thick with a microkeratome (surgical corneal knife). Medication has been given the patient to cause the iris (colored portion of the eye—part 43) to retract; therefore, the surgeon;can see the natural lens (44), which rests in a capsule (much like a lung or kidney, but thinner—part 46). For ease of presentation, FIG. 23 omits showing the natural lens; however, the surgical process described requires the surgeon to make an opening in the natural lens capsule and remove the natural lens A small anterior capsule aperture (47) is started about 2 to 3 millimeters from the anterior apex of the natural lens capsule (46). This aperture is continued in a circular pattern until a 360 degree section has been removed (50). Next the natural lens and any scattered remains are removed from the eye.

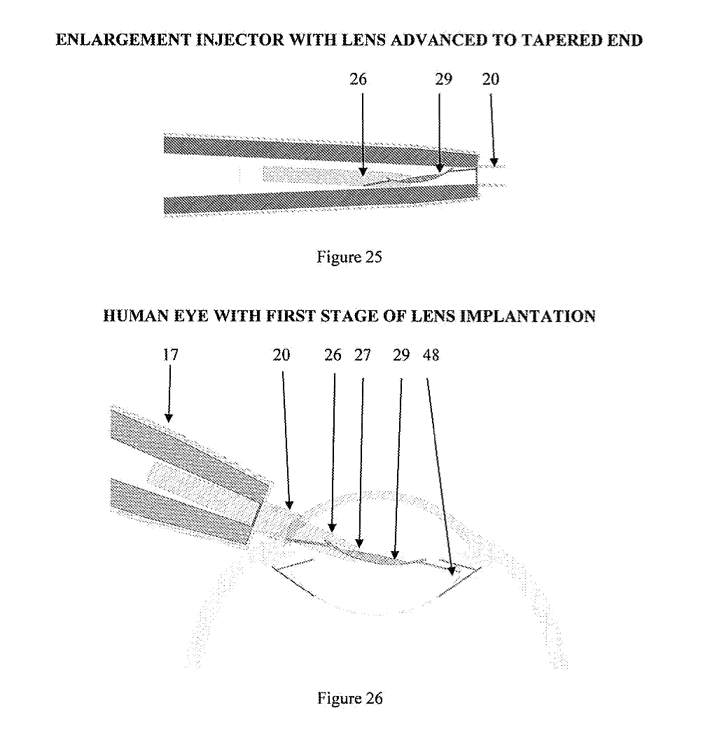

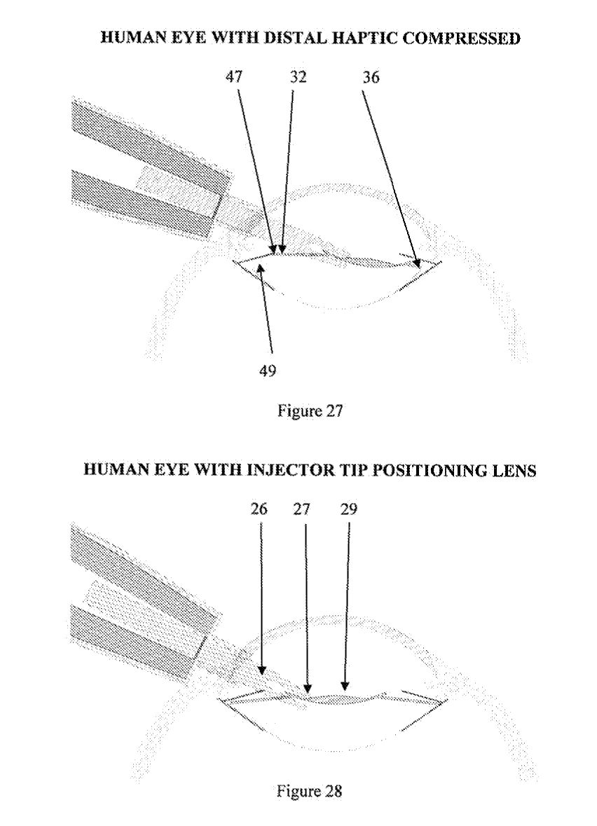

The surgeon assures the lens haptic distal tip (FIG. 24—part 36) is visible in the tip of the injector over sleeve (20). FIG. 25 is an enlarged view of the lens (29) in the injector. Now the surgeon places the lens injector over sleeve extension (FIG. 26—part 20) preferably about 2 mm into the incision (40) made between the cornea (41) and sclera (42) and advances the injector tip (26) moving the lens (29) into the over sleeve tip (20). The slot (21) will open and the lens will move into the space left by the removal of the crystalline lens through the incision (40) in the eye. The over sleeve extension slot (21) opens ant the lens passes through the over sleeve extension (20) and into the eye. The gap (FIG. 27—part 28) in the injector rod tip (26) is so designed to be used by the surgeon to guide the lens distal haptic (36) into the capsule (46) that contained the natural lens. Continuing to gently push the lens will cause the distal haptic (36) to slightly collapse much like a spring under tension. The process continues by stretching the distal portion of the natural lens until the proximal haptic (32) has 8.5 mm or more clearance for placement into the proximal end of the anterior capsule aperture (47) and into the proximal anterior portion (49) of the natural lens capsule. Next (FIG. 28) the tip of the injector rod (26) is rotated slightly allowing both surfaces of the tip to contact both surfaces of the lens (34). The rotation continues until a slight pressure is achieved, then the injector rod tip (26) is retracted until the center of the lens optic (35) is aligned with the center of the cornea.

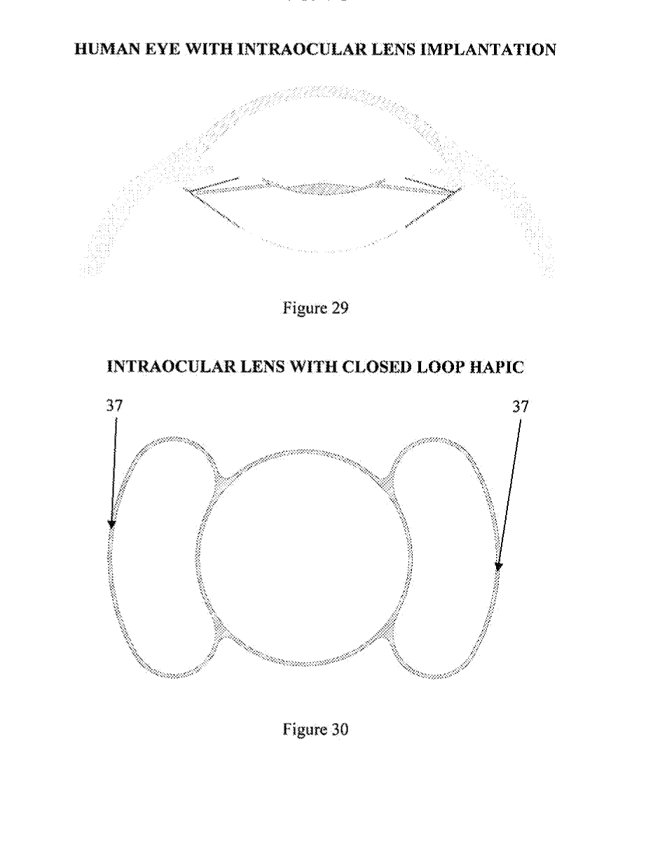

The injector tip is now rotated to remove the slight pressure between the tip and lens and removed from the eye. FIG. 29 shows the lens in position within the eye at the completion of the surgery.

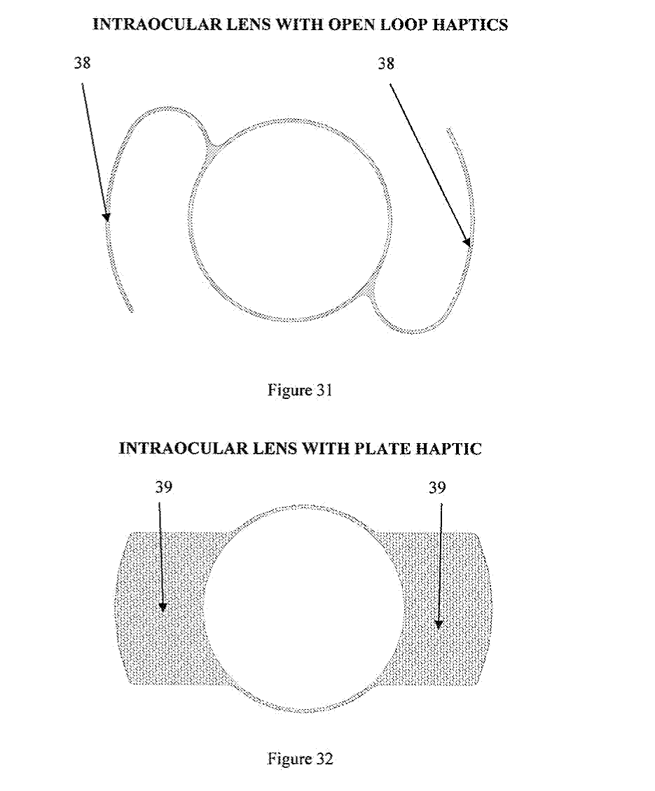

While the surgery and injector are described using a closed loop haptic as shown in FIG. 30, the process works with open looped haptics such as the example shown in FIG. 31. The system also works well with a plate haptic as shown in FIG. 32. The injector contact end gap (28) length and breath are adjusted to accommodate these alternative haptic designs as well as any lens optics with a center apex greater than 475 microns.

The following examples illustrate embodiments of the invention, but should not be viewed as limiting the scope of the invention.

### Examples

FIG. 1 depicts an injector barrel (1) that is approximately 114 millimeters long. The:portion that is not tapered (cylindrical part—2) is approximately 50 millimeters long and the tapered portion (3) is 64 millimeters long. The cylindrical end view of the injector (4) can be seen in FIG. 2. The major axis (8) is curved at the end at the end to form the side walls (5) of the minor axis (9). The lens (FIG. 3) shows an overhead view of an intraocular lens (29) where the widest dimension (30) of the lens (29) is along the haptic (32 & 36). In other models the widest point may be along the periphery (34) of the optic. The widest dimension (haptic or optic) perpendicular to the long axis (31) of the haptics is smaller than the major axis (8) of the injector opening. The minor axis (9) is large enough to allow the lens to move during sterilization, but not large enough to allow the lens to turn over; therefore, if placed into the injector correctly the lens will remain in the proper position for implantation. FIG. 4 is a sagittal view of the same lens (29) where the proximal (32) and distal (36) haptics are shown. The maximum thickness of the lens occurs at the apex (35) of the lens. Radially outward from the center section (35) is a mid section (33) that is connected to the thinnest section (34); which can be less than 100 microns.

In FIG. 5 the minor axis (9) has remained constant while most of the reduction of the internal dimensions of the injector has occurred along the major axis (10) to where the shape is approaching or has become a portion of a circle that made up the curved side walls (5) of the minor axis.

FIG. 6 shows the stopper on the large end. The stopper has a portion (13) that is shaped to fit tightly into the bore of the injector. The outer diameter (14) of the larger stopper fits into a second commercial vial suitable for sterilization of a medical device allowing stability between the injector-container and the outer vial. FIG. 7 shows the stopper with the tapered section (15) for the distal end of the injector. The outer diameter (16) functions to stabilize the injector and the outer vial and is approximately the same diameter as the outer diameter (14) of the stopper placed in the end of the cylindrical end (4) of the injector.

FIG. 8 shows a barrel over sleeve (17) that can be made of a plastic with the injector barrel (1) placed into the over sleeve after the tapered end stopper (15) is removed. The over sleeve has a tapered section (FIG. 9—part 19) that fits against the end of the tapered end (12) of the injector barrel (1). The over sleeve extension (20) has a slot (21) which permits the tip to be smaller than the incision in the eye and the over sleeve tip inserted without resistance and expand to the dimensions of the compressed lens as the lens is passed through the tapered over sleeve tip (22).

FIG. 10 shows the injector rod (23) that is inserted into the large end (4) of the injector barrel (1). The outer end of the injector rod has a thumb position (24) to press the injector rod through the injector barrel (1). A compression gasket (25), located some distance from the thumb position (24), allows the fluids (viscoelastic material) in the injector to assist in pushing the lens through the injector. FIG. 11 shows the assembly ready to be inserted into the injector barrel (1). The distal tip (26) of the injector rod (23) is enlarged in FIG. 12. The tip leading edge (27) is designed to allow easy insertion of the lens. A gap(28) is cut into the tip to allow the tip of the injector to rest at a position along the lens optic as to prevent the tip from increasing the cross sectional area of the lens to interfere with the minor axis (9) of the injector at the apex of the lens (35). FIG. 13 shows the injector tip (26) with the proximal haptic (32) bent to the posterior side of the injector tip where the lens thinner section (34) is in the injector rod tip gap (28) and the injector rod tip leading edge (27) rest against the lens mid section (33).

FIG. 14 depicts the lens optic outer (34) section with clearance between the injector barrel large end minor axis side walls (5). The widest portion of the lens in the example is the haptic and there is no compression as the distance between apexes of the minor axis walls (5) is greater than the widest point (FIG. 3 part 30) of the lens, which is the position of the lens for sterilization.

FIG. 15 shows the lens has moved to where the major axis has decreased to where the minor axis sidewalls are contacting the thinner portion of the lens optic. FIG. 16 shows the cross sectional view of the lens partially compressed by reducing the major axis of the barrel (lumen) by approximately 800 microns. In FIG. 17 only the outer portion of the optic (34) is bent. FIGS. 18 to 21 show flexing of the lens components until the lens is folded to where most of the open air space has been eliminated; however, none of the water within the lens has been squeezed out of the material.

FIG. 22 shows the cross section of a typical human eye before removing the natural lens (44). Also shown is the sclera (42), the white milky looking section that surrounds the cornea (41) that is clear and the part of the eye that initially bends light entering the eye. For surgically opening the eye an incision (40) is made along the junction between the sclera and cornea. A cataract is a fogging of the natural lens (44) of the eye. The natural lens is contained in a capsule (46) with a structure much like a thin kidney or lung and is positioned just behind the iris (43) the colored portion of the eye. The apex (45) of the natural lens is the center of the anterior curve of the natural lens. FIG. 23 shows an eye with the natural lens removed after a tear (47) was started 2 to 3 millimeters from the apex of the natural lens capsule and torn into a circle (47 & 50). The opening in the natural lens capsule is used toy remove the cataract and implant the intraocular lens.

FIG. 24 shows a lens (29) in an injector advanced to where the surgeon is ready to inject the lens (29) and FIG. 25 shows an enlarged view where the lens (29) can be seen in the injector rod tip (26) and just short of entering the over sleeve extension (20).

FIG. 26 shows the lens (29) during initial injection into the eye and being placed into the distal posterior capsule of the natural lens (48), while the advancement of the lens to compress the distal haptic (36) is shown in FIG. 27 as is the positioning of the proximal haptic (32) near the proximal opening (47) in the anterior capsule for placement into proximal portion (49) of the capsule. FIG. 28 shows the lens (29) is centered using the tip of the injector (27) and the finish surgery is shown in FIG. 29.

FIG. 30 is a model of a closed loop haptic, where the haptic is connected at both ends to the optic. With the open loop haptic of FIG. 31 shows only one end of the haptic attached to the optic; therefore, it is a cantilever design. The plate haptic of FIG. 32 is more like the closed loop haptic except the insides of the loop are solid.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, where ever used, is intended to include the terms consisting and consisting essentially of. It is intended that the specification and examples be considered exemplary only with the true scope and spirit of the invention indicated by the following claims.

TABLE

| PART | |
|---|---|
| NUMBER | PART NAME |
| 1 | Injector Barrel |
| 2 | Cylindrical portion |
| 3 | Tapered portion |
| 4 | Large end ovoidal opening |
| 5 | Minor axis side walls - portion of a circle |
| 6 | Centers of. the radii of the minor axis sides |
| 7 | Side walls of major axis |
| 8 | Major axis of the large end opening |
| 9 | Minor axis which remains constant throughout the injector |
| | barrel |
| 10 | Major axis at the distal tapered end become smaller |
| 11 | Outside diameter |
| 12 | Last 6 millimeters of outside section of tapered end |
| 13 | Portion of large stopper that plugs into the large end (2) |
| 14 | Stopper insert diameter on the large, (straight) end |
| 15 | Tapered portion of small end stopper that presses onto the |
| | barrel |
| 16 | Stopper diameter on the tapered (small) end of the injector |
| 17 | Over sleeve |
| 18 | Finger support |
| 19 | Tapered section |
| 20 | Extension |
| 21 | Extension slot |
| 22 | Extension taper |
| 23 | Injector rod |
| 24 | Thumb position |
| 25 | Compression gasket |
| 26 | Contact End located at the leading end (distal) of the |
| | injector rod |
| 27 | Contact End leading edge |
| 28 | Contact End gap |
| 29 | Intraocular lens |
| 30 | Widest point in plane perpendicular to long axis of haptics |
| 31 | Longest length of the lens along axis of haptics |
| 32 | Hapties - proximal |
| 33 | Optic middle section |
| 34 | Optic outer portion (thinnest) |
| 35 | Optic center section with apex which is the thickest part |
| 36 | Distal Haptic where the tip of the haptic initially enters |
| | the injector |
| 37 | Closed loop haptic |
| 38 | Open loop haptic |
| 39 | Plate haptic |
| | Eye |
| 40 | Incision for the removal of a cataract and the implantation |
| | of an intraocular lens |
| 41 | Cornea - clear portion of the eye that transmits and refracts |
| | light toward the natural lens |
| 42 | Sclera - the white milky portion of the eye that surrounds |
| | the cornea |
| 43 | Iris - colored portion of the eye - changes shape or size to |
| | meter the light entering the eye |
| 44 | Natural crystalline lens |
| 45 | Anterior apex |
| 46 | Natural lens capsule |
| 47 | With anterior capsule aperture |
| 48 | Distal (posterior) part |
| 49 | Proximal portion |
| 50 | Capsular rexis - 360 degree tear in capsule |

1. An intraocular lens injector assembly comprising:
an injector barrel having a circular first end and a tapered second end; and
an intraocular lens within the barrel;
wherein the assembly is comprised of a material that, is packaged and sterilized as a single unit.

2. The assembly of claim 1, wherein the material is a glass, a plastic or a combination thereof, and the material is capable of being steam or gas sterilized.

3. The assembly of claim 1, wherein the material is sterilizable in accordance with at least one government regulation.

4. The assembly of claim 1, wherein the barrel is a straight cylindrical section of a length and width that permits the intraocular lens freedom of movement but prevents the lens from turning upside down.

5. The assembly of claim 1, wherein a cross section of the barrel is in the shape of an ovoid at the circular first end.

6. The assembly of claim 1, wherein a cross section of the barrel is in the shape of a circle at the tapered second end.

7. The assembly of claim 1, wherein the longitudinal axis of the intraocular lens is coaxial with the longitudinal axis of the barrel.

8. The assembly of claim 1, wherein a cross section of the circular first end of the barrel is an ovoid which reduces in size toward the tapered second end and becomes circular.

9. The assembly of claim 1, further comprising a stopper which is coupled to the circular first end of the barrel.

10. The assembly of claim 1, further comprising a stopper which is coupled to the tapered second end of the barrel.

11. The assembly of claim 1, further comprising a vial.

12. The assembly of claim 1, further comprising an over sleeve surrounding the barrel.

13. The assembly of claim 12, wherein the over sleeve comprises finger grips along one side.

14. The assembly of claim 12, wherein the over sleeve is tapered.

15. The assembly of claim 12, wherein the over sleeve further comprises an extension portion.

16. The assembly of claim 15, wherein the extension portion is tapered to allow one end of the extension portion to penetrate an eye.

17. The assembly of claim 12, wherein the extension portion comprises a slot along one side or a plurality of slots along two sides.

18. The assembly of claim 1, further comprising a rod within the barrel.

19. The assembly of claim 18, wherein the rod comprises a thumb pad at a first end.

20. The assembly of claim 18, further comprising a compression gasket attached to the rod.

21. The assembly of claim 20, wherein the compression gasket functions to aid movement of the intraocular lens into the tapered end of the barrel.

22. The assembly of claim 18, wherein the rod comprises two protrusions at a second end.

23. The assembly of claim 22, wherein the two protrusions are parallel to the longitudinal axis of the rod.

24. The assembly of claim 22, wherein the two protrusions are offset to the longitudinal axis of the rod.

25. The assembly of claim 22, further comprising a gap between the two protrusions.

26. The assembly of claim 25, wherein the gap is sufficiently wide to allow the intraocular lens to pass through the gap.

27. The assembly of claim 26, wherein the gap is of sufficient size to allow the tips to rest against the lens optic when used with a plate lens.

28. The assembly of claim 22, wherein the two protrusions comprise tips that rest against the intraocular lens and near the apex of the intraocular lens.

29. The assembly of claim 28, wherein the gap is of sufficient size to allow the tips to rest against the intraocular lens optic when used with an open looped lens.

30. The assembly of claim 28, wherein the gap is of sufficient size to allow the tips to rest against the intraocular lens optic when used with a closed loop lens.

31. The assembly of claim 28, wherein the longitudinal axis of the two protrusions and the longitudinal axis of the gap are parallel to the longitudinal axis of the barrel.

32. The assembly of claim 22, wherein the two protrusion comprise tips that rest away from the apex of the intraocular lens so as not to increase the cross sectional area of the combination of the cross sections of the intraocular lens and the two protrusions.

33. The assembly of claim 32, wherein the tips aid in moving the intraocular lens through the injector.

34. The assembly of claim 22, wherein the two protrusions aid in manipulating the intraocular lens when placed within an eye.

35. The assembly of claim 22, wherein the width of the two protrusions is smaller than the width of the barrel.

36. A method for inserting an intraocular lens into an eye of a patient comprising:
providing a sterilized assembly composed of an injector barrel which has an ovoid or circular first end and a tapered second end, an intraocular lens within the barrel, a rod within the barrel wherein the rod has a thumb pad at one end and two protrusions at the other end;
extending the tapered seconded end into the eye;
pushing on the thumb pad to move the lens through the barrel toward the tapered second end and into the eye; and
inserting the intraocular lens into the eye.

37. A method of sterilizing an intraocular lens injector assembly comprising an injector barrel having a circular first end and a tapered second end; and an intraocular lens within the barrel, comprising subjecting the assembly as a single unit to a steam or gas sterilization cycle.
